(19) United States
(12) Patent Application Publication
(10) Pub. No.: US 2010/0292705 A1
(43) Pub. Date: Nov. 18, 2010

(54) Sizing Guide for Femoral Prosthesis

(75) Inventor: Asit Shah, Ridgewood, NJ (US)

Correspondence Address: Ryder, Lu, Mazzeo & Konieczny LLC, PO Box 387, CONSHOHOCKEN, PA, 19428-0387, US

(73) Assignee: MAXX ORTHOPEDICS, INC., Plymouth Meeting, PA (US)

(21) Appl. No.: 12/465,045

(22) Filed: May 13, 2009

(51) Int. Cl. A61B 17/58

(52) U.S. Cl. 606/102

(57) ABSTRACT

A posterior-referencing apparatus for sizing the resected distal femur prior to implanting the femoral component of a total knee prosthesis. The apparatus can be used for sizing either the left or right femur. The apparatus has a first operable orientation adapted for sizing the left femur and a second operable orientation offset 180 degrees from the first operable position for sizing the right femur. The apparatus can be reconfigured between left and right orientations quickly and easily without disassembling the apparatus.

### FIELD OF THE INVENTION

The invention relates to an apparatus for measuring the anterior posterior dimensions of either the left or right resected distal femur in order to select the properly-sized femoral component of a total knee prosthesis.

### BACKGROUND OF THE INVENTION

Total knee replacement surgery is well-known in the prior art and is becoming more commonplace every day. Total knee replacement surgery involves implantation of complimenting and articulating femoral and tibial components to the distal femur and proximal tibia, respectively.

To implant a total knee prosthesis, the distal femur must first be prepared to receive the femoral component of the knee prosthesis. Typically, the distal femur must be cut along multiple planes to establish accurately-located surfaces to which the femoral component is connected. The first transverse cut can be made using various known distal femoral cutting guides, such as disclosed by applicant in co-pending U.S. patent application Ser. No. 12/198,439 entitled Distal Femoral Cutting Guide, filed Aug. 26, 2008, incorporated herein by reference. The next five cuts can be made by using various known additional cutting guides, which are usually located and secured to the transverse, resected surface created during initial resection. The next four cuts include an axially-directed anterior cut, an axially-directed posterior cut, an angled cut, an anterior chamfer and a posterior chamfer.

The precise size and location of the final five cuts depend on the size and shape of the femoral component, which is typically provided in a variety of sizes. Prior to making the final five cuts, the surgeon must select the particular component size that will best fit the requirements of a particular implant site. Once the particular femoral component is selected, it must be located and oriented so as to attain appropriate rotational alignment and create a symmetric flexion gap.

A variety of femoral sizing guides are known in the prior art for assisting in the selection of a standard-size femoral knee prosthesis that will best fit the requirements of a particular implant site. The sizing guide also assists in locating the corresponding femoral cutting guide appropriately on the transverse distal femoral surface.

A number of patents have issued that disclose instruments and/or jigs for sizing the resected distal femur. To accommodate the asymmetrical shape of the distal femur, such devices have complex designs that require time-consuming reconfiguration when the device is switched from the left to the right femur, and vice versa. Alternatively, the surgeon may use separate devices that are specially configured for use on either the left femur or the right femur. However, the added cost of purchasing, maintaining and sterilizing two sizing guides is undesirable. Therefore, it would be desirable to provide a single device that can be used to size both the left and the right resected distal femur, which does not require complex or time-consuming reconfiguration.

### SUMMARY OF THE INVENTION

The invention comprises a posterior-referencing apparatus for sizing the resected distal femur prior to implanting the femoral component of a total knee prosthesis. The apparatus can be used for sizing either the left or right femur. The apparatus has a first operable orientation adapted for sizing the left femur and a second operable orientation offset 180 degrees from the first operable position for sizing the right femur. The apparatus can be reconfigured between left and right orientations quickly and easily without disassembling the apparatus.

The apparatus generally comprises a femoral sizing block, a stylus pivotally connected to the sizing block, a stabilizer, and means for adjustably connecting the stabilizer to the sizing block in first and second configurations without disconnecting any components of the apparatus. The first configuration is operatively arranged for installation on the left femur and the second configuration is operatively arranged for installation on the right femur.

The sizing block has opposed medial and lateral surfaces, opposed anterior and posterior surfaces, and opposed distal and proximal locator surfaces arranged to contact the transverse, resected distal femoral surface. One of the distal and proximal locator surfaces is arranged to contact the resected distal surface of the right femur and the other locator surface is arranged to contact the resected distal surface of the left femur. A central axis extends between the anterior and posterior surfaces and is symmetrically located relative to the lateral and medial surfaces and the distal and proximal locator surfaces. A plurality of guide bores extends between the distal and proximal surfaces.

The stabilizer has opposed medial and lateral feet arranged to engage the posterior surface of the medial and lateral condyles, respectively, when the apparatus engages the resected distal femoral surface. The stylus has a proximal end pivotally connected to the anterior surface of the sizing block and a distal end arranged to engage the anterior surface of the distal femur.

The means for adjustably connecting the stabilizer and the sizing block enables rotation of the sizing block about the central axis between the first and second configurations and releasably locks the sizing block in one of the configurations. In a preferred embodiment, the connecting means comprises a central bore co-axial with the central axis and extending between the anterior and posterior surfaces of the sizing block, and a post connected at one end to the stabilizer and extending through the central bore in the sizing block.

In a preferred embodiment, the post has a first portion having a first cross-sectional profile that compliments the profile of the bore. The complimenting profiles enable translation of the sizing block along the post but prevent rotation of the sizing block around the post. The post has a second portion having a second cross-sectional profile different than the first profile. The second profile enables translation of the sizing block along the post and enables rotation of the sizing block around the post.

The post includes a stop fixed to the upper end which prevents the stabilizer from disassembling from the post. The post also includes a plurality of size indicating indicia along its length that align with a reference point on the stabilizer block.

The apparatus further includes means for locking the sizing block on the first portion of the post and preventing translation along the post. In a preferred embodiment, the locking means comprises first and second threaded bores extending from the distal and proximal locator surfaces, respectively, to the central bore, and a threaded fastener arranged to engage either of the bores and abut the post.

### BRIEF DESCRIPTION OF THE DRAWINGS

FIG. 1 is an exploded perspective of a sizing guide in accordance with a preferred embodiment of the invention;

FIG. 2 is a side elevation of a sizing guide installed on a resected distal femur;

FIG. 3 is a top plan view of the apparatus in a first configuration, shown in solid lines, and a second configuration, shown in dashed lines; and,

FIG. 4 is an exploded, sectional, front plan view of the sizing block and post of the apparatus of FIG. 1.

### BRIEF DESCRIPTION OF PREFERRED EMBODIMENTS

For the purpose of illustration, an embodiment of the invention is shown in the accompanying drawings. However, it should be understood by those of ordinary skill in the art that the invention is not limited to the precise arrangements and instrumentalities shown therein and described below. Throughout the specification, like reference numerals are used to designate like elements. Numerous changes and modifications within the spirit and scope of the invention will become apparent to those skilled in the art from this detailed description.

Unless otherwise defined, all technical and scientific terms used herein in their various grammatical forms have the same meaning as commonly understood by one of ordinary skill in the art to which this invention belongs. The terms anterior, posterior, proximal, distal, medial, lateral, and transverse are used herein with their conventional medical/anatomical meaning as defined, for example, in Dorland's Illustrated Medical Dictionary. When used in connection with the apparatus, the terms proximal and distal reference the surgeon using the apparatus. In contrast, when used in connection with the femur with which the apparatus is used, the terms proximal, distal, anterior and posterior reference the patient.

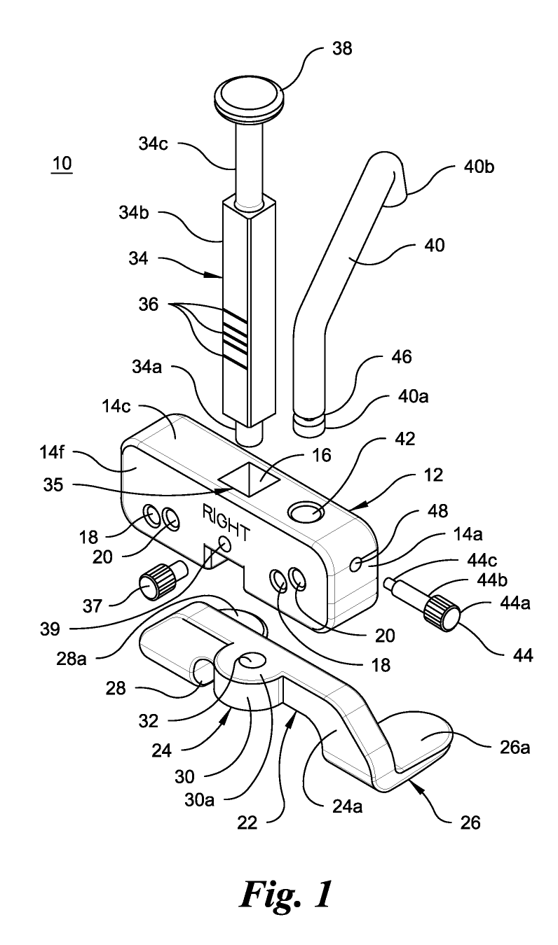

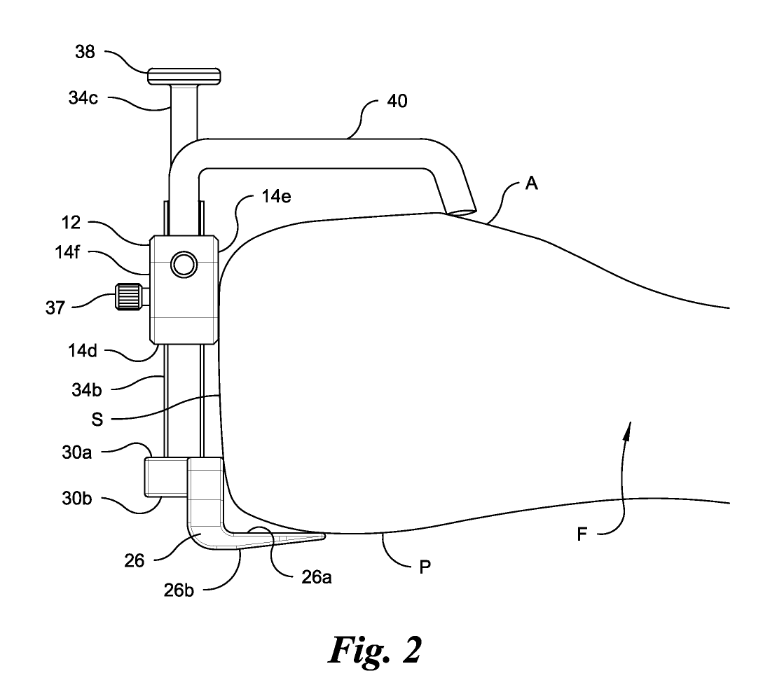

An apparatus in accordance with an embodiment of the invention is illustrated in FIGS. 1-4 and is designated generally by reference numeral 10. The apparatus 10 is constructed and arranged to help a surgeon select the proper size of a standard femoral component of a total knee prosthesis to be implanted on a resected distal femur. As best seen in FIG. 2, the apparatus is constructed and arranged to engage the transverse resected surface “S” and posterior condylar surface “P” of the femur “F” to facilitate the selection of the femoral component from a plurality of standard size components.

As described below in greater detail, the apparatus 10 is adapted for use on either the left or right resected femur of a patient by making a simple reconfiguration without disassembly of the components. For purposes of description and illustration, the apparatus 10 is shown in a first configuration adapted for installation on the right femur and the terms distal, proximal, medial and lateral, as applied to the sizing block 12, are used with reference to the first configuration. However, it should be understood to those of ordinary skill in the art that the apparatus 10 is changed to its second configuration adapted for installation on the left femur by rotating the sizing block 180 degrees about the post 34. In the second configuration, the distal and lateral surfaces transpose positions with the proximal and medial surfaces, respectively.

The sizing apparatus 10 includes a sizing block having opposed medial 14a and lateral 14b surfaces, opposed anterior 14c and posterior 14d surfaces, and opposed distal 14e and proximal 14f locator surfaces. The locator surfaces 14e, 14f are arranged to abut the resected distal surface S of the femur when the apparatus 10 is properly installed on the femur.

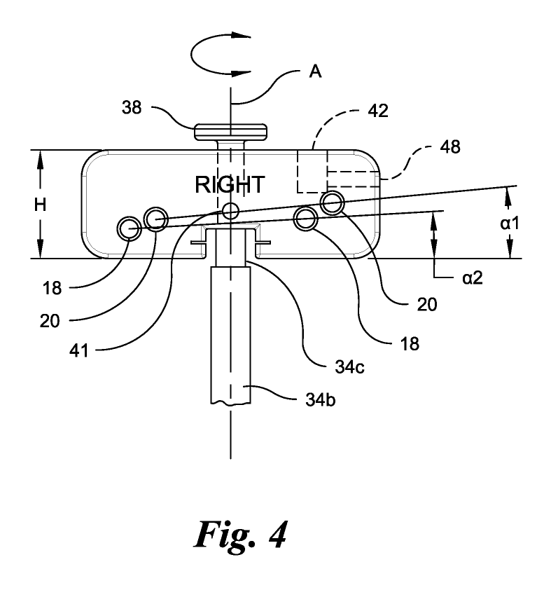

With the exception of a single stylus bore 42 and key bore 48 located on only one side thereof, and the angular orientation of the guide bores 18, 20 relative to the epicondylar axis, the sizing block 12 has a generally-symmetrical construction about a central axis “A”, which extends between the anterior 14c and posterior 14d surfaces. Referring to FIG. 4, the central axis “A” is symmetrically located relative to the medial 14a and lateral 14b surfaces and the distal 14e and proximal 14f locator surfaces.

A central bore 16 extends between the anterior 14c and posterior 14d surfaces of the sizing block 12. The bore 16 is coaxial with the central axis and has a generally-square cross-sectional profile. The profile of the bore 16 compliments the profile of the central portion 34b of the post 34 (described below). The bore 16 could have a different cross-sectional profile, such as rectangular, without departing from the scope of the invention.

A plurality of guide bore pairs 18, 20 extend between the distal 14e and proximal 14f surfaces. The guide bores 18 locate mounting pins, not shown, which are inserted through the bores and then driven into the femur. The pins are subsequently used to locate a femoral cutting guide, which will later be used to make the subsequent resections to the femur in preparation of receiving the femoral component. Multiple bore pairs 18, 20 are preferably provided, which are arranged at slightly different angles relative to the epicondylar axis so that the subsequent femoral cutting guide can be oriented at different angles on the femur. For example, referring to FIG. 4, the first bore pair 18 is oriented at an angle of approximately 5 degrees relative to horizontal as reflected by angle α1, whereas the second bore pair 20 is oriented at an angle of approximately 3 degrees as reflected by angle α2. When the sizing block 12 is rotated and reconfigured from the right to left orientation, the bores 18, 20 accommodate the asymmetrical shape of the condyles.

A stabilizer 22 is mounted under the sizing block. In a preferred embodiment, the stabilizer 22 comprises a yoke 24 having integrally-formed, opposed medial and lateral feet 26, 28, which extend outwardly generally-transverse to the yoke 24 and transverse to the distal 14e and proximal 14f locator surfaces. As best seen in FIG. 2, the upper surface 26a, 26b of the feet are generally-flat and adapted to engage the posterior surface “P” of the medial and lateral condyles, respectively, when the sizing block 12 engages the resected distal femoral surface “S”. The yoke 24 is symmetrical and can engage either the left or the right femur in the same manner.

The yoke 24 has a central hub 30 formed on its proximal face 24a. The hub 30 has a central bore 32 extending there through from its anterior surface 30a to its posterior surface 30b. The hub bore 32 has a size and shape that compliments the lower tab portion 34a of the post 34 to connect the post 34 to the stabilizer 22. In the embodiment shown in FIGS. 1-4, the hub bore 32 is cylindrical and extends entirely through the hub 30.

A measuring post 34 extends through and slidably engages the central bore 16 in the sizing block 12. In the embodiment shown in FIGS. 1-4, the post 34 comprises an elongate bar having a lower tab portion 34a, central sizing portion 34b, and an upper reconfiguring portion 34c.

The lower tab portion 34a has a cylindrical shape with dimensions that compliment the bore 32 in the hub 30. The diameter of the tab portion 34a is slightly smaller than the diameter of the bore 32, and the length of the lower portion 34a is about equal to the depth of the bore 32 so that the tab 34a snugly engages the bore 32 without overlap. In this embodiment, the post 34 is fixed to the stabilizer yoke 24 by spot welding the tab to the posterior surface 30b of the hub 30 with the lower shoulder of the post contacting the anterior surface 30a of the hub 30.

The central sizing portion 34b has a square shape with dimensions that compliment the central bore 16 in the sizing block 12. The sizing portion 34b is slightly smaller than the central bore 16 so that the sizing portion can freely and smoothly slide within the bore 16.

The central sizing portion 34b includes a plurality of size indicating indicia 36 along its length that align with a reference point 35 on the sizing block 12. The size indicating indicia 36 correspond to one of a standard-size femoral prosthetic component. The surgeon uses the measuring post reading, in conjunction with other intra-operative observations, to select the femoral component.

The upper reconfiguring portion 34c has a cylindrical shape with dimensions that allow the sizing block 12 to rotate about the measuring post 34 when the sizing block 12 is translated upward until the central bore 16 is aligned with the reconfiguring portion 34c as seen in FIG. 4. For example, the length of the reconfiguring portion 34c is slightly larger than the height “H” of the sizing block, whereas the outer diameter of the reconfiguring portion 34c is smaller than the width of the central bore 16 in the sizing block 12.

A handle 38 is fixed to the upper end of the post 34. In the embodiment shown in FIGS. 1-4, the handle 38 is disc-shaped and can be comfortably grasped by the surgeon. The diameter of the handle 38 is larger than the central bore 16 in the sizing block 12 so that the sizing block 12 cannot translate upwardly beyond the end of the post 34. In this regard, the handle 38 also functions as a stop to prevent disassembly of the components.

Optionally, a set screw 37 releasably locks the post 34 in the central bore 16. The set screw 37 engages a threaded bore 39, which extends through the sizing block 12 from the proximal locator surface 14f to the central bore 16. The sizing block 12 has a similarly sized and located bore 41 on the opposite side, shown in FIG. 4, through which the set screw extends when the apparatus is used in the left configuration.

A stylus 40 is rotatably mounted in a bore 42 on the anterior side of the sizing block 12. The diameter of the stylus 40 and bore 42 are chosen so that the stylus 40 freely and smoothly rotates within the bore 42 without wobbling. To enable rotational movement within but prevent upward translational movement out of the bore 42, a key 44 engages a radial groove 46 in the bottom of the stylus 40. As best seen in FIG. 1, the key 44 has an enlarged-diameter, knurled knob at the proximal end 44a, a threaded central shank portion 44b, and a reduced-diameter distal end 44c. The key 44 engages a threaded bore 46 extending from the medial surface 14a of the sizing block 12 to the stylus bore 42. When fully inserted into the bore 46, the distal end 44c of the set screw 44 enters the radial groove 46 but does not impinge on it.

As best seen in FIG. 2, the stylus 40 is U-shaped and extends upwardly and outwardly from the sizing block 12. When the apparatus is installed on the resected femur, the stylus can be pivotally adjusted until the distal end 40b of the stylus 40 contacts the anterior surface of the femur.

In a preferred embodiment, the components are made from a solid material that is durable and can be sterilized in an autoclave. For example, the apparatus components may be made from type 302 stainless steel.

The apparatus 10 can be easily installed on a resected femur by first loosening the set screw 37, which allows the sizing block to translate freely along the central portion 34b of the measuring post 34. The distal locator surface 14e of the sizing block 12 is then applied against the resected femoral face “S” while the stabilizer feet 26 are brought into contact with the posterior condylar surface “P”. Next, a fine adjustment is made by pivoting the stylus 40 until it contacts the low point on the anterior condyl. To lock the apparatus on the femur, a set screw 37 can optionally be installed in the bore 39 then tightened against the measuring post 34. Pins are inserted through the appropriate guide bores 18, 20 and driven into the resected surface S, which are subsequently used to locate a femoral cutting guide. The surgeon then removes the apparatus, examines the size indicia 36, and selects a standards-size femoral component based on the reading.

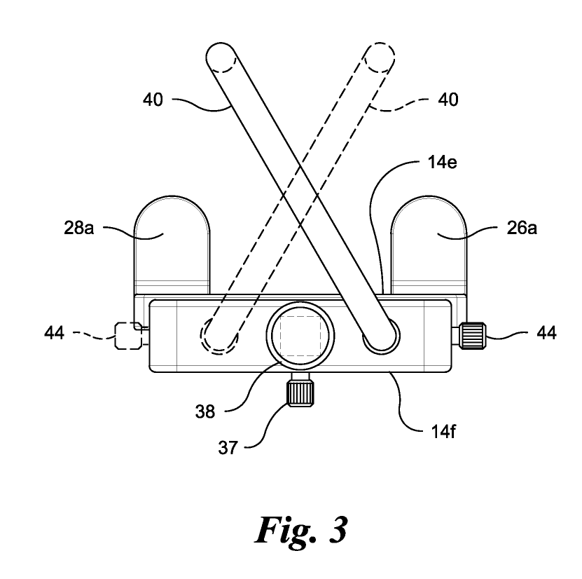

The apparatus can be quickly and easily reconfigured for use on the opposite (left) femur. First, the set screw 37 (if installed) is loosened to allow the sizing block 12 to translate freely on the post 34. The sizing block 12 is then slid upwardly until it overlaps the upper reconfiguring portion 34c as seen in FIG. 4. In this position, the sizing block 12 can be rotated freely relative to the sizing post 34. The sizing block 12 should be rotated 180 degrees and then slid back down onto the central portion 34b of the measuring post 34. The apparatus 10 can then be installed on left femur in the same manner as described above. A comparison of the left and right configuration is shown in FIG. 3.

1. An apparatus for determining the size of the femoral prosthetic component to be implanted on either the left or right femur having a transverse resected distal femoral surface, comprising:
a) a femoral sizing block having:
i) opposed medial and lateral surfaces, opposed anterior and posterior surfaces, and opposed distal and proximal locator surfaces arranged to contact the transverse, resected distal femoral surface,
ii) a central axis extending between the anterior and posterior surfaces and symmetrically located relative to the lateral and medial surfaces and the distal and proximal locator surfaces;
iii) a plurality of guide bores extending between the distal and proximal surfaces;
b) a stabilizer having opposed medial and lateral feet arranged to engage the posterior surface of the medial and lateral condyles, respectively, when the apparatus engages the resected distal femoral surface;
c) a stylus having a proximal end pivotally connected to the anterior surface of said block and a distal end arranged to engage the anterior surface of the distal femur;
d) means for adjustably connecting said stabilizer and said sizing block in first and second configurations without disconnecting any of said components, the first configuration operatively arranged for installation on the left femur and the second configuration operatively arranged for installation on the right femur.

2. The apparatus recited in claim 1, wherein one of said distal and proximal locator surfaces is arranged to contact the resected distal surface of the right femur and the other of said distal and proximal locator surfaces is arranged to contact the resected distal surface of the left femur.

3. The apparatus recited in claim 2, wherein said connecting means enables rotation of the sizing block about the central axis between the first and second configurations and releasably locks the sizing block in said configuration.

4. The apparatus recited in claim 1, wherein said connecting means comprises:
a) a central bore co-axial with said central axis and extending between the anterior and posterior surfaces of the sizing block; and,
b) a post connected at one end to said stabilizer and extending through said central bore in said sizing block.

5. The apparatus recited in claim 4, wherein said post comprises:
a) a first portion having a first cross-sectional profile that compliments the profile of the bore, said complimenting profiles enabling translation of said sizing block along the post but preventing rotation of the sizing block around the post; and,
b) a second portion having a second cross-sectional profile different than the first profile, said second profile enabling translation of the sizing block along the post and enabling rotation of the sizing block around the post.

6. The apparatus recited in claim 5, wherein said post includes a stop fixed to the other end which prevents the stabilizer from disengaging the post.

7. The apparatus recited in claim 6, wherein said post includes a plurality of size indicating indicia along its length that align with a reference point on the stabilizer block.

8. The apparatus recited in claim 6, including means for locking said sizing block on the first portion of said post and preventing translation along the post.

9. The apparatus recited in claim 8, wherein said locking means comprises first and second threaded bores extending from the distal and proximal locator surfaces, respectively, to the central bore, and a threaded fastener arranged to engages either of said bores and abut the post.

10. The apparatus recited in claim 6, including means for locking said stylus and preventing rotation on the sizing block.
